(19) United States
(12) Patent Application Publication
(10) Pub. No.: US 2007/0027426 A1
(43) Pub. Date: Feb. 1, 2007

(54) Iontophoresis device to deliver active agents to biological interfaces

(75) Inventors: Takehiko Matsumura, Shibuya-ku (JP); Mizuo Nakayama, Shibuya-ku (JP); Hidero Akiyama, Shibuya-ku (JP); Akihiko Tanioka, Ota-ku (JP); Kiyoshi Kanamura, Hachiji-shi (JP)

Correspondence Address: SEED INTELLECTUAL PROPERTY LAW GROUP PLLC, 701 FIFTH AVE, SUITE 5400, SEATTLE, WA, 98104, US

(73) Assignee: Transcutaneous Technologies Inc., Shibuya-ku (JP)

(21) Appl. No.: 11/475,838

(22) Filed: Jun. 26, 2006

Related U.S. Application Data
(60) Provisional application No. 60/693,668, filed on Jun. 24, 2005.

(51) Int. Cl. A61N 1/30

(52) U.S. Cl. 604/20

(57) ABSTRACT

An iontophoresis device includes an active electrode assembly which comprises an active electrode element and an outermost active electrode ion selective membrane that caches an active agent. The outermost active electrode ion selective membrane may be formed by one or more ion exchange membranes. The active electrode assembly may also comprise an electrolyte and/or one or more inner active electrode ion selective membranes. The inner active electrode ion selective membrane may be a “leaky” ion selective membrane. The inner active electrode ion exchange membrane may be spaced from the outermost active electrode ion selective membrane, for example, by one or more non-ion selective porous membranes or by a buffer material and/or buffer reservoir. An iontophoresis device may also include a counter electrode assembly and/or voltage source.

### CROSS-REFERENCE TO RELATED APPLICATION

This application claims the benefit under 35 U.S.C. § 119(e) of U.S. Provisional Patent Application No. 60/693,668, filed Jun. 24, 2005.

### BACKGROUND

1. Field

This disclosure generally relates to the field of iontophoresis and, more particularly, to the delivery of active agents such as therapeutic agents or drugs to a biological interface under the influence of electromotive force.

2. Description of the Related Art

Iontophoresis employs an electromotive force to transfer an active agent such as an ionic drug or other therapeutic agent to a biological interface such as skin or mucus membrane.

Iontophoresis devices typically include an active electrode assembly and a counter electrode assembly, each coupled to opposite poles or terminals of a voltage source, such as a chemical battery. Each electrode assembly typically includes a respective electrode element to apply an electromotive force. Such electrode elements often comprise a sacrificial element or compound, for example silver or silver chloride.

The active agent may be either cationic or anionic, and the voltage source can be configured to apply the appropriate voltage polarity based on the polarity of the active agent. Iontophoresis may be advantageously used to enhance or control the delivery rate of the active agent. As discussed in U.S. Pat. No. 5,395,310, the active agent may be stored in a reservoir such as a cavity, or stored in a porous structure or as a gel. Also as discussed in U.S. Pat. No. 5,395,310, an ion exchange membrane may be positioned to serve as a polarity selective barrier between the active agent reservoir and the biological interface.

Commercial acceptance of iontophoresis devices is dependent on a variety of factors, such as cost to manufacture, shelf-life or stability during storage, efficiency of active agent delivery, safety of operation, and disposal issues. An iontophorsis device that addresses one or more of these factors, and further related advantages is desirable.

### BRIEF SUMMARY

In one aspect, the present disclosure is directed to an iontophoresis device operable for delivering one or more active agents to a biological interface such as skin, mucous membranes, and the like. The iontophoresis device includes an active electrode element, an outermost active electrode ion selective membrane, one or more active agents of a first polarity, and a first inner active electrode ion selective membrane.

The one or more active agents of a first polarity may be loaded in the interstices of the outermost active ionic selective membrane and substantially retained therein in the absence of an electromotive force and transferred outwardly from the outermost active electrode ion selective membrane in the presence of an electromotive force. The outermost active electrode ion selective membrane may be spaced from the active electrode element, and the outermost active electrode ion selective membrane may include a plurality of interstices. The inner active electrode ion selective membrane may be positioned between the electrolyte and the outermost active electrode ion selective membrane. The first inner active electrode ion selective membrane characterized in that in the presence of an electromotive force of the first polarity, the first inner active electrode ion selective membrane passes ions of a polarity that is opposite to the first polarity and that have a size approximately less than a threshold size, and in that in the presence of the electromotive force of the first polarity the first inner active electrode ion selective membrane limits passage of ions of a polarity that is the same as the first polarity such that a fraction of a total charge flux across the first inner active electrode ion selective membrane that is attributable to the ions of the first polarity is in the range from about 0.05 to about 0.5 of the total charge flux across the first inner active electrode ion selective membrane

The outermost active-electrode ion selective membrane may comprise one or more ion exchange membranes, which may further include ion exchange groups that temporarily bonds or caches the active agent. The leaky first inner active electrode ion selective membrane may be spaced from the outermost active electrode ion selective membrane, for example, by a porous membrane. The iontophoresis device may further include a counter electrode assembly and a voltage source electrically coupled between the active and the counter electrode assemblies.

In another aspect, the present disclosure is directed to an iontophoresis device operable to deliver active agents to a biological interface. The iontophoresis device includes an active electrode element, an outermost active electrode ion selective membrane spaced from the active electrode element, the outermost active electrode ion selective membrane comprising a plurality of interstices, a first inner active electrode ion selective membrane positioned between the active electrode element and the outermost active electrode ion selective membrane and spaced from the outermost active electrode ion selective membrane to form at least one space therebetween, and an active agent of a first polarity loaded in the interstices of the outermost active ionic selective membrane and substantially retained therein in the absence of an electromotive force and transferred outwardly from the outermost active electrode ion selective membrane in the presence of an electromotive force. The iontophoresis device may also include a first porous membrane spacing the first inner active electrode ion selective membrane from the outermost active electrode ion selective membrane. The iontophoresis device may also include a substitutive buffer material positioned between the outermost active electrode ion selective membrane and the first inner active electrode ion selective membrane, the substitutive buffer material including ions having a polarity that matches a polarity of the active agent.

### BRIEF DESCRIPTION OF THE SEVERAL VIEWS OF THE DRAWINGS

In the drawings, identical-reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1A is a block diagram of an iontophoresis device comprising active and counter electrode assemblies according to one illustrated embodiment where the active electrode assembly includes an outermost active electrode ion selective membrane that caches an active agent, an active electrode element, an electrolyte reservoir, an inner active electrode ion selective membrane and a buffer solution reservoir.

FIG. 1B is a block diagram of an iontophoresis device comprising active and counter electrode assemblies according to one illustrated embodiment where the active electrode assembly includes an outermost active electrode ion selective membrane caching an active agent formed by three ion exchange membranes, an active electrode element, an electrolyte reservoir, an inner active electrode ion selective membrane and a substitutive buffer material that supplies ions to substitute for the active agent in the outermost active electrode ion selective membrane and that spaces the inner active electrode ion selective membrane from the outermost active electrode ion selective membrane.

FIG. 2A is a block diagram of an iontophoresis device comprising active and counter electrode assemblies according to one illustrated embodiment where the active electrode assembly includes an outermost active electrode ion selective membrane that caches an active agent, an active electrode element, an electrolyte reservoir, an inner active electrode ion selective membrane, and a spacer that spaces the inner active electrode ion selective membrane from the outermost active electrode ion-selective membrane.

FIG. 2B is a block diagram of an iontophoresis device comprising active and counter electrode assemblies according to one illustrated embodiment where the active electrode assembly includes an outermost active electrode ion selective membrane is formed by three ion exchange membranes that caches an active agent, an active electrode element, an electrolyte reservoir, an inner active electrode ion selective membrane, and a spacer that spaces the inner active electrode ion selective membrane from the outermost active electrode ion selective membrane.

FIG. 3A is a block diagram of an iontophoresis device comprising active and counter electrode assemblies according to one illustrated embodiment where the active electrode assembly includes an outermost active electrode ion selective membrane that caches an active agent, an active electrode element, an electrolyte reservoir, an inner active electrode ion selective membrane.

FIG. 3B is a block diagram of an iontophoresis device comprising active and counter electrode assemblies according to one illustrated embodiment where the active electrode assembly includes an outermost active electrode ion selective membrane that caches an active agent formed by three ion exchange membranes, an active electrode element, an electrolyte reservoir, an inner active electrode ion selective membrane.

FIG. 4A is a block diagram of an iontophoresis device comprising active and counter electrode assemblies according to one illustrated embodiment where the active electrode assembly includes an outermost active electrode ion selective membrane that caches an active agent, an active electrode element, an electrolyte reservoir, a first inner active electrode ion selective membrane spaced from ion selective membrane by a first non-ion selective porous membrane and a second inner active electrode ion exchange spaced from the first inner ion exchange membrane by a second non-ion selective porous membrane.

FIG. 4B is a block diagram of an iontophoresis device comprising active and counter electrode assemblies-according-to one-illustrated embodiment where the active electrode assembly includes an outermost active electrode ion selective membrane that caches an active agent formed by three ion exchange membranes, an active electrode element, an electrolyte reservoir, a first inner active electrode ion selective membrane spaced from ion selective membrane by a first non-ion selective porous membrane and a second inner active electrode ion exchange spaced from the first inner ion exchange membrane by a second non-ion selective porous membrane.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with iontophoresis device controllers including but not limited to voltage and/or current regulators have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is, as “including, but not limited to.”

Reference throughout this specification to “one embodiment” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Further more, the particular features, structures, or characteristics may be combined in any-suitable manner in one or more embodiments.

It should be noted that, as used in this specification and the appended claims, the singular forms “a,” “an,” and “the” include plural referents unless the content clearly dictates otherwise. Thus, for example, reference to an iontophoresis device including “an active electrode element ”includes a single active electrode element, or two or more active electrode elements. It should also be noted that the term “or” is generally employed in its sense including “and/or” unless the content clearly dictates otherwise.

The headings provided herein are for convenience only and do not interpret the scope or meaning of the embodiments.

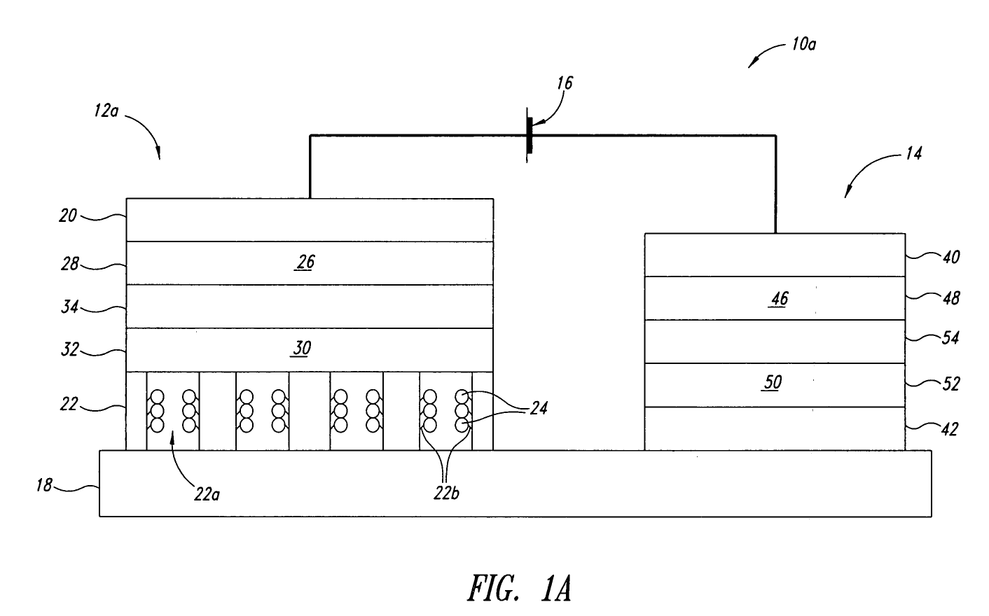

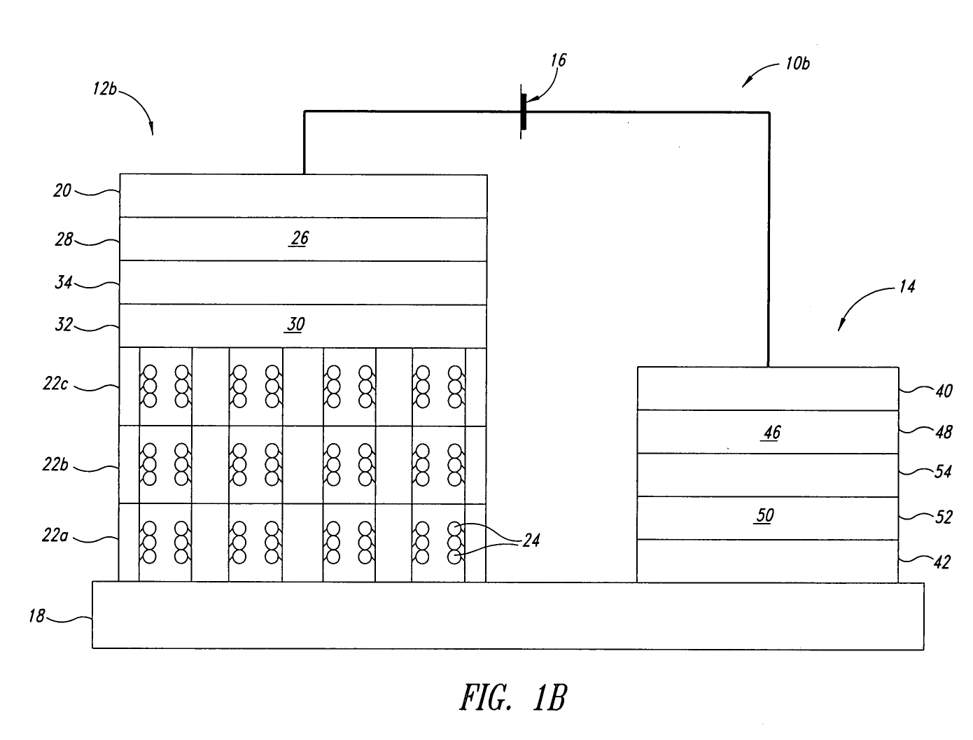

FIG. 1A shows an iontophoresis device 10a comprising active and counter electrode assemblies 12a, 14, respectively, electrically coupled to a voltage source 16 to supply an active agent 24 to a biological interface 18, such as a portion of skin or mucous membrane via iontophoresis, according to one illustrated embodiment.

The active electrode assembly 12a comprises an active electrode element 20 electrically coupled to a first pole having a first polarity (e.g., positive, negative) of the voltage source 16 and positioned in the active electrode assembly 12a to apply an electromotive force to transport the active agent 24 via various other components of the active electrode assembly 12a. The active electrode element 20 may take a variety of forms. For example, the active electrode element 20 may include a sacrificial element such as a chemical compound or amalgam including silver (Ag) or silver chloride (AgCl). Such compounds or amalgams typically employ one or more heavy metals, for example lead (Pb), which may present issues with regard manufacturing, storage, use and/or disposal. Consequently, some embodiments may advantageously employ a non-metallic active electrode element. Such may, for example, comprise multiple layers, for example a polymer matrix comprising carbon and a conductive sheet comprising carbon fiber or carbon fiber paper, such as that described in commonly assigned pending Japanese patent application 2004/317317, filed Oct. 29, 2004.

The active electrode assembly 12a comprises an outermost active electrode ion selective membrane 22, generally opposed across the active electrode assembly 12a from the active electrode element 20. The outermost active electrode ion selective membrane 22 may take a variety of forms. For example, the outermost active electrode ion selective membrane 22 may take the form of a charge selective ion exchange membrane such as a cation exchange membrane or an anion exchange membrane, which substantially passes and/or blocks ions based primarily on the charge carried by the ion. Suitable ion exchange membranes are commercially available from a variety of sources. For example, cation exchange membranes are available under the designators NEOSEPTA, CM-1, CM-2, CMX, CMS, and CMB from Tokuyama Co., Ltd. Also for example, anion exchange membranes are available under the designators NEOSEPTA, AM-1, AM-3, AMX, AHA, ACH, and ACS also from Tokuyama Co., Ltd.

The outermost active electrode ion selective membrane 22 advantageously temporarily retains or caches the active agent 24, for example a drug or therapeutic agent, proximate the biological interface 18 while an electromotive force is not being applied by the active electrode element 20, and releases or transfers the active agent 24 while an electromotive force is being applied by the active electrode element 20. Thus, the active agent 24 may be preloaded in the outermost active electrode ion selective membrane 22 before any electromotive force is applied, and stored or cached until placed in use. Preloading may be accomplished, for example, via diffusion, by soaking the outermost active electrode ion selective membrane 22 in a solution with a high concentration of the active agent 24. Repeated soaking may produce a higher concentration of active agent 24 in the outermost active electrode ion selective membrane 22. When placed in use, the active agent 24 is transferred outwardly from the outermost active electrode ion selective membrane 22 to an exterior portion of the active electrode assembly 12a that is in contact with the biological interface 18. The active agent 24 may include therapeutic agents, pharmaceutical compositions, therapeutic drugs, and the like. Examples of the active agent 24 include epinephrine, fentanyl, lidocaine, pilocarpine, nonsteroidal anti-inflammatory drugs (NSAIDS), corticosteroids, and the like, or pharmaceutical salts thereof. In at least one embodiment, the active agent 24 may take the form of lidocaine hydrochloride or lidocaine hydrochloride (2%) with epinephrine (0.00125%).

In particular embodiments, the active agent 24 may be bonded to ion exchange groups 22b in the cavities or interstices 22a of the outermost active electrode ion selective membrane 22. This advantageously caches the active agent 24 proximate, and in some embodiments, directly in contact with the biological interface 18, which may increase transport efficiency and/or increase the speed of delivery.

Alternatively, the outermost active electrode ion selective membrane 22 may take the form of a semi-permeable membrane that substantially passes and/or blocks ions based on a size or molecular weight of the ion. In such an embodiment, other methods, structures or properties may be used to retain the active agent 24 in the outermost active electrode ion selective membrane 22. For example, a release liner may retain the active agent in the outermost active electrode ion selective membrane 22 until the liner is removed for use. Alternatively, the active agent 24 may be retained in the outermost active electrode ion selective membrane 22 in a dehydrated or dry state, where the hydration of the active agent 24 permits such to move under the influence of an electromotive force.

Whether the outermost active electrode ion selective membrane 22 takes the form of an ion exchange membrane or a semi-permeable membrane, the outermost active electrode ion selective membrane 22 may take a variety of physical forms. The outermost active electrode ion selective membrane 22 may take the form a solid, liquid-or gel. The-outermost active-electrode ion selective membrane 22 may, for example, take the form a material with a distinct lattice structure such as a polymer, or a material without a distinct lattice structure.

In use, the outermost active electrode ion selective membrane 22 may be placed directly in contact with the biological interface 18. Alternatively, an interface coupling medium (not shown) may be employed between the outermost active electrode ion selective membrane 22 and the biological interface 18. The interface coupling medium may, for example, take the form of an adhesive and/or gel. The gel may, for example, take the form of a hydrating gel. If used, the interface coupling medium should be permeable by the active agent 24.

The active electrode assembly 12a also comprises an electrolyte 26 positioned between the active electrode element 20 and the outermost active electrode ion selective membrane 22, proximate the active electrode element 20. The electrolyte 26 may provide ions or donate charges to prevent or inhibit the formation of gas bubbles (e.g., hydrogen) on the active electrode element 20 in order to enhance efficiency, and to prevent or inhibit the formation of acids or bases (e.g., H⁺ ions, OH⁻ ions) or neutralize the same, which may enhance efficiency, increase delivery rates, and/or reduce the potential for irritation of the biological interface 18. As discussed in further below, in some embodiments the electrolyte 26 may provide ions or donate charges to compensate for the exiting active agent in the outermost active electrode ion selective membrane 22, for example substituting for the active agent 24 bonded to the ion exchange groups 22b where the outermost active electrode ion selective membrane 22 takes the form of an ion exchange membrane. Such may facilitate transfer of the active agent 24 to the biological interface 18, for example, increasing and/or stabilizing delivery rates. A suitable electrolyte may take the form of a solution of 0.5 M disodium fumarate: 0.5 M Poly acrylic acid (5:1).

In other embodiments, the electrolyte 26 may provide ions having an identical structure to that of the active agent 24. For example, in some embodiments where the active agent 24 comprises ionized-lidocaine and/or epinephrine, the electrolyte 26 may include ionized lidocaine and epinephrine, or salts thereof. In certain other embodiments, the electrolyte 26 may include one or more salts of a drug, therapeutic agent, and the like, with the identical structure as that of the active agent 24 loaded in the interstices 22a of the outermost counter electrode ion selective membrane 22.

The electrolyte 26 may be retained by an electrolyte reservoir 28, for example where the electrolyte 26 is in solution form. The electrolyte reservoir 28 may take a variety of forms capable of temporarily retaining an electrolyte 26. For example, the electrolyte reservoir 28 may take the form of a cavity formed by one or more membranes, a porous membrane or a gel.

The active electrode assembly 12a illustrated in FIG. 1 additionally comprises a substitutive buffer material 30 disposed between electrolyte 26 and the outermost active electrode ion selective membrane 22. The buffer material 30 may supply ions to substitute for the active agent 24 in the outermost active electrode ion selective membrane 22, as the active agent 24 is transferred from the outermost active electrode ion selective membrane 22 to the biological interface 18. Such substitution may improve efficiency and/or may increase and/or stabilize delivery rate to the biological interface 18. Consequently, the substitutive buffer material 30 may advantageously comprise, for example, a salt (e.g., NaCl) and/or a vitamin (e.g., B12a solution).

The buffer material 30 may be temporarily retained by a buffer reservoir 32. The buffer reservoir 32 may take a variety of forms capable of temporarily retaining the buffer material 30. For example, the buffer reservoir 32 may take the form of a cavity defined by one or more membranes, a porous membrane and/or a gel.

The active electrode assembly 12a may further comprise an inner active electrode ion selective membrane 34 positioned between and/or to separate, the electrolyte 26 from the buffer material 30. The inner active electrode ion selective membrane 34 is spaced from the outermost active electrode ion selective membrane 22 by the buffer material 30 and/or buffer reservoir 32. Such spacing may advantageously eliminate or reduce electrolysis of water which may occur at an interface between the two membranes 34, 22. This elimination or reduction in electrolysis may in turn inhibit or reduce the formation of acids and/or bases (e.g., H⁺ ions, OH⁻ ions), that would otherwise present possible disadvantages such as reduced efficiency, reduced transfer rate, and/or possible irritation of the biological interface 18.

The inner active electrode ion selective membrane 34 may take a variety of forms, for example, a charge selective ion exchange membrane in some embodiments, a molecular weight or size selective semi-permeable membrane in other embodiments, or a “leaky” charge selective ion exchange membrane in still further embodiments.

Typical charge selective ion exchange membranes allow the preferential transport of either cations (e.g., cation exchange membranes) or anions (e.g., anion exchange membranes). The transport number (transference number) of an ion represents the fraction of current carried by that ion. The sum of the transport numbers for the anions and the cations equals 1. The transport number of the preferentially transported ion in typical charge selective ion exchange membranes is usually about 1. In the case of a “leaky” charge selective ion exchange membrane, the term “leaky” may refer to a membrane having a transport number of the preferentially transported ion of less that 1. The term “threshold size” refers to the maximum particle (e.g., ion) size the may diffuse through the membrane.

In an embodiment, the first inner active electrode ion selective membrane 34 characterized in that in the presence of an electromotive force of the first polarity, the first inner active electrode ion selective membrane 34 passes ions of a polarity that is opposite to the first polarity and that have a size approximately less than a threshold size, and in that in the presence of the electromotive force of the first polarity the first inner active electrode ion selective membrane 34 limits passage of ions of a polarity that is the same as the first polarity such that a fraction of a total charge flux across the first inner active electrode ion selective membrane 34 that is attributable to the ions of the first polarity is in the range from about 0.05 to about 0.5 of the total charge flux across the first inner active electrode ion selective membrane. In another embodiment, the first inner active electrode ion selective membrane 34 is further characterized in that in the absence of the electromotive force of the first polarity the fraction of total charge flux across the first inner active electrode ion selective membrane 34 attributable to ions of the first polarity is essentially zero.

In another embodiment, the first inner active electrode ion selective membrane 34 characterized in that in the presence of an electromotive force of the first polarity, the first inner active electrode ion selective membrane 34 passes ions of a polarity that is opposite to the first polarity and that have a size approximately less than a threshold size, and in that in the presence of the electromotive force of the first polarity the first inner active electrode ion selective membrane limits passage of ions of a polarity that is the same as the first polarity such that passage of ions of the first polarity from the electrolyte across the first inner active electrode ion selective membrane 34 is approximately equal to or greater than a flux of the one or more active agents of the first polarity across the outermost active electrode ion selective membrane 22. In another embodiment, the first inner active electrode ion selective membrane 34 is further characterized in that in the absence of the electromotive force of the first polarity a total flux across the first inner active electrode ion selective membrane 34 attributable to ions of the first polarity is essentially zero.

Whether the inner active electrode ion selective membrane 34 takes the form of an ion exchange membrane, a semi-permeable membrane or a “leaky” ion exchange membrane, the inner active electrode ion selective membrane 34 may take a variety of physical forms. The inner active electrode ion selective membrane 34 may take the form a solid, liquid or gel. The inner active electrode ion selective membrane 34 may, for example, take the form a material with a distinct lattice structure such as a polymer, or a material without a distinct lattice structure.

With respect to a “leaky” charge selective ion exchange membrane embodiment, the inner active electrode ion selective membrane 34 substantially freely allows passage of ions of opposite charge to the charge of the active agent 24 at a first rate, yet allows passage of ions of the same charge as that carried by the active agent 24 at a second rate, lower than the first rate. This may be the results of the pore size of the ion exchange membrane being sufficiently large as to allow a transfer of anions with a molecular size smaller than the threshold size, and/or the number of ion exchange groups being sufficiently small that some of the ions of the same charge as the active agent 24 leaking or leaching through the ion exchange membrane.

Thus, where the active agent 24 is a cationic drug or therapeutic agent, the outermost active electrode ion selective membrane 22 will pass cations outwardly, and the inner active electrode ion selective membrane 34 may take the form of an anion exchange membrane, selective to pass negatively charged ions inwardly from the buffer material 30 to the electrolyte 26. However, the anion exchange membrane will allow some passage of positively charged ions, such as sodium ions, from the electrolyte 26 toward the outermost active electrode ion selective membrane 22. Such ions may substitute for the active agent 24 in the outermost active electrode ion selective membrane 22, or in the substitutive buffer material 30.

On the other hand, where the active agent 24 is an anionic drug or therapeutic agent, the outermost active electrode ion selective membrane 22 will pass anions outwardly, and the inner active electrode ion selective membrane 34 may take the form of a cation exchange membrane, selective to pass positively charged ions inwardly from the buffer material 30 to the electrolyte 26. However, the cation exchange membrane will allow some passage of negatively charged ions, such as chloride ions, from the electrolyte 26 toward the outermost active electrode ion selective membrane 22. Such ions may substitute for the active agent 24 in the outermost active electrode ion selective membrane 22, or in the substitutive buffer material 30.

Iontophoresis generally uses a direct current of either positive or negative polarity to transfer drugs of the corresponding polarity into the skin. The amount of current applied over a period of time determines the amount of drug delivered and is usually expressed as milliampere per minute (mAmin). For example, applying a current (I) of 4 mA for a time (T) of 10 minutes corresponds to a 40 mAmin dose. Using Faraday's law, the amount of drug deliver (D) can be determine by the relationship D=(IT)/ZF, where I is the current, T is the time, Z is the valance of the drug and F is Faradays constant. For example, applying a current of 4 mA for 10 minutes to a drug having a valance of (⁺1) corresponds to a theoretical delivery rate of about 3×10² nmol•min⁻¹. Applying a current of 1 mA for 10 minutes to a drug having a valance of (⁺1) corresponds to a theoretical delivery rate of about 6×10² nmol•min⁻¹.

In an embodiment, the first inner active electrode ion selective membrane 34 characterized in that in the presence of an electromotive force of the first polarity, the first inner active electrode ion selective membrane 34 passes ions of a polarity that is opposite to the first polarity and that have a size approximately less than a threshold size, and in that in the presence of the electromotive force of the first polarity the first inner active electrode ion selective membrane 34 limits passage of ions of a polarity that is the same as the first polarity such that passage of ions of the first polarity from the electrolyte 26 across the first inner active electrode ion selective membrane 34 ranges from about 600 nmol·min⁻¹ to about 3000 nmol·min⁻¹ when a flux of the one or more active agents 24 of the first polarity across the outermost active electrode ion selective membrane 22 ranges from about 600 nmol·min⁻¹ to about 3000 nmol·min⁻¹.

In at least one embodiment, the one or more active agents 24 may take the form of lidocaine hydrochloride or lidocaine hydrochloride (2%) with epinephrine (0.00125%). When ionized, lidocaine and epinephrine each carry a positive charge. In such embodiment, the first inner active electrode ion selective membrane 34 may take the form of an anion exchange membrane (AEM) characterized in that in the presence of an electromotive force having a positive polarity, the first inner active electrode ion selective membrane 34 passes ions of negative polarity that have a size approximately less than a threshold size, and in that in the presence of the electromotive force having a positive polarity, the first inner active electrode ion selective membrane 34 limits passage of ions of positive polarity such that passage of the positive ions from the electrolyte 26 across the first inner active electrode ion selective membrane 34 ranges from about 600 nmol·min⁻¹ to about 3000 nmol·min⁻¹ when a flux of the one or more active agents 24 of a positive polarity across the outermost active electrode ion selective membrane 22 ranges from about 600 nmol·min⁻¹ to about 3000 nmol·min⁻¹.

The counter electrode assembly 14 comprises a counter electrode element 40 electrically coupled to a second pole of the voltage source 16, the second pole having an opposite polarity to the first pole. The counter electrode element 40 may take a variety of forms, for example the sacrificial electrode element (e.g., silver or silver chloride) or non-sacrificial element (e.g., carbon) discussed above. The counter electrode assembly 14 allows completion of an electrical path between poles of the voltage source 16 via the active electrode assembly 12a and the biological interface 18.

The counter electrode assembly 14 may also comprise an outermost counter electrode ion selective membrane 42. The outermost counter electrode ion selective membrane 42 may take a variety of forms. For example, the outermost counter electrode ion selective membrane 42 may take the form of a charge selective ion exchange membrane, such as a cation exchange membrane or an anion exchange membrane, which substantially passes and/or blocks ions based on the charge carried by the ion. Examples of suitable ion exchange membranes are discussed above. Alternatively, the outermost counter-electrode ion selective membrane 42 may take the form of a semi-permeable membrane that substantially passes and/or blocks ions based on size or molecular weight of the ion.

In some embodiments the outermost counter electrode ion selective membrane 42 may be positioned in the counter electrode assembly 14 so as to be in direct contact with the biological interface 18 when placed in use. Alternatively, an interface coupling medium (not shown) may be employed between the outermost counter electrode ion selective membrane 42 and the biological interface 18. The interface coupling medium may, for example, take the form of an adhesive and/or gel. The gel may, for example, take the form of a hydrating gel.

The outermost counter electrode ion selective membrane 42 of the counter electrode assembly 14 is selective to ions with a charge or polarity opposite to that of the outermost active electrode ion selective membrane 22 of the active electrode assembly 12a. Thus, for example, where the outermost active electrode ion selective membrane 22 of the active electrode assembly 12a transfers negatively charged ions of the active agent 24 to the biological interface 18, the outermost counter electrode ion selective membrane 42 of the counter electrode 14 transfers positively charged ions to the biological interface 18. On the other hand, where the outermost active electrode ion selective membrane 22 of the active electrode assembly 12a transfers positively charged ions of the active agent 24 to the biological interface 18, the outermost counter electrode ion selective membrane 42 of the counter electrode assembly 14 transfers negatively charged ions to the biological interface 18.

The counter electrode assembly 14 also comprises an electrolyte 46 positioned between the counter electrode element 40 and the outermost counter electrode ion selective membrane 42, proximate the counter electrode element 40. The electrolyte 46 may provide ions or donate charges to prevent or inhibit the formation of gas bubbles (e.g., hydrogen) on the counter electrode element 40, and/or may prevent or inhibit the formation of acids or bases or neutralize the same, which may enhance efficiency and/or reduce the potential for irritation of the biological interface 18. The electrolyte 46 may be retained by an electrolyte reservoir 48, for example where the electrolyte 46 is in solution form. The electrolyte reservoir 48 may take a variety of forms capable of temporarily retaining an electrolyte 46. For example, the electrolyte reservoir 48 may take the form of a cavity formed by one or more membranes, a porous membrane and/or a gel.

The counter electrode assembly 14 additionally comprises a buffer material 50 disposed between electrolyte 46 and the outermost counter electrode ion selective membrane 42. The buffer material 50 may supply ions for transfer through the outermost counter electrode ion selective membrane 42 to the biological interface 18. Consequently, the buffer material 50 may, for example, comprise a salt (e.g., NaCl). The buffer material 50 may be temporarily retained by a buffer reservoir 52. The buffer reservoir 52 may take a variety of forms capable of temporarily retaining the buffer material 50. For example, the buffer reservoir 52 may take the form of a cavity formed by one or more membranes, a porous membrane or a gel.

The counter electrode assembly 14 further comprises an inner counter electrode ion selective membrane 54 positioned between and/or to separate, the electrolyte 46 from the buffer material 50. The inner counter electrode ion selective membrane 54 may take a variety of forms. For example, the inner counter electrode ion selective membrane 54 may take the form of a substantially charge selective ion exchange membrane, allowing passage of ions of opposite charge to those passed by the outermost counter electrode ion selective membrane 42. Alternatively, the inner counter electrode ion selective membrane 54 may the form of a molecular weight or size selective membrane such as a semi-permeable membrane. The inner counter electrode ion selective membrane 54 may prevent transfer of undesirable elements or compounds into the buffer material 50. For example, the inner counter electrode ion selective membrane 54 may prevent of the transfer of hydrogen (H⁺) or sodium (Na⁺) ions from the electrolyte 46 into the buffer material 50.

The voltage source 16 may take the form of one or more chemical battery cells, super- or ultra-capacitors, or fuel cells. The voltage source 16 may, for example, provide a voltage of 12.8V DC, with tolerance of 0.8V DC, and a current of 0.3 mA. The voltage source 16 may be selectively electrically coupled to the active and counter electrode assemblies 12a, 14 via a control circuit, for example, via carbon fiber ribbons. The iontophoresis device 10a may include discrete and/or integrated circuit elements to control the voltage, current and/or power delivered to the electrode assemblies 12a, 14. For example, the iontophoresis device 10a may include a diode to provide a constant current to the electrode elements 20, 40.

As suggested above, the active agent 24 may take the form of an ionizable, cationic, or an anionic drug or other therapeutic agent, or pharmaceutical salts thereof. Consequently, the poles or terminals of the voltage source 16 may be reversed. Likewise, the selectivity of the outermost ion selective membranes 22, 42 and inner ion selective membranes 34, 54 may be reversed.

FIG. 1B shows an iontophoresis device 10b according to another illustrated embodiment. This embodiment, and other embodiments described herein, is substantially similar in some respects to the previously described embodiments. Hence, common structures and acts are identified by the same reference numbers. Only significant differences between the structure and operation of the various embodiments are described below.

In the embodiment illustrated in FIG. 1B, the outermost active electrode ion selective membrane 22 of the active electrode assembly 12b takes the form of multiple charge selective membranes, such as the three ion exchange membranes 22a, 22b, 22c. As noted above, ion exchange membranes such as cation exchange membranes or anion exchange membranes are commercially available. The use of multiple ion exchange membranes 22a, 22b, 22c may permit a larger dose or amount of the active agent 24 to be loaded into the active electrode assembly 12b.

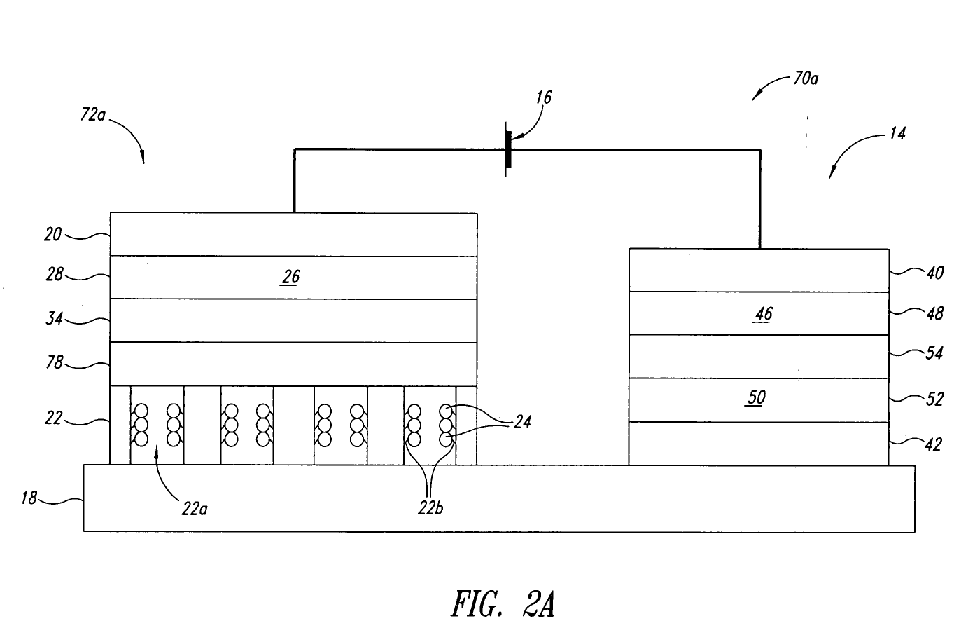

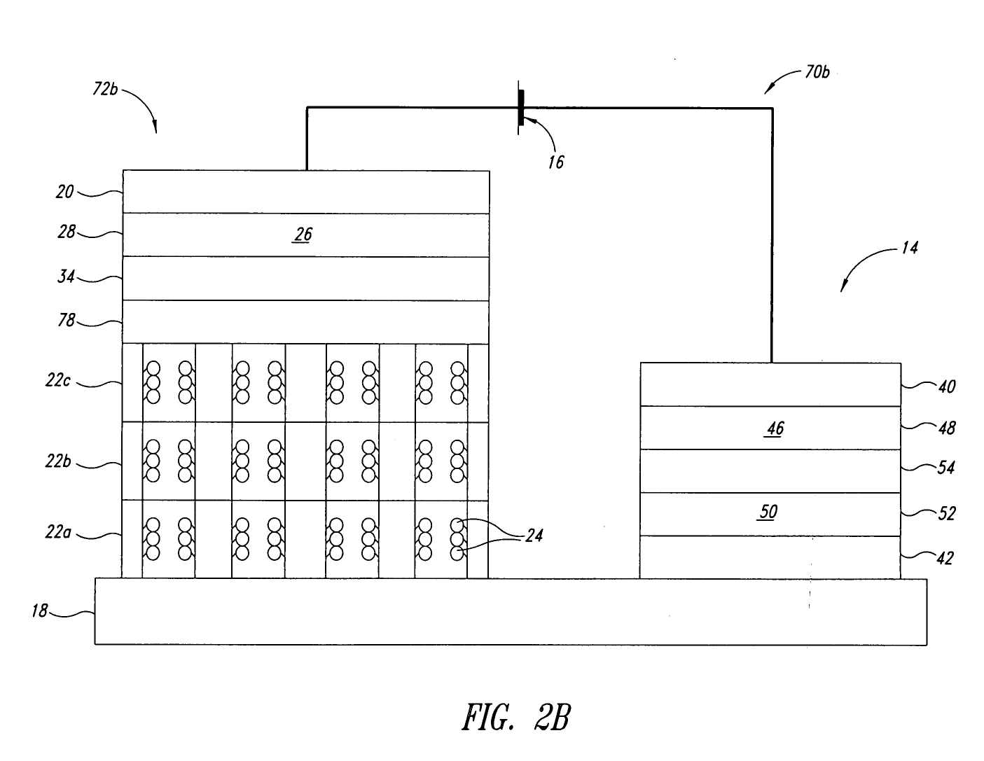

FIG. 2A shows an iontophoresis device 70a according to another illustrated embodiment. This embodiment, and other embodiments described herein, is substantially similar in some respects to the previously described embodiments. Hence, common structures and acts are identified by the same reference numbers. Only significant differences between the structure and operation of the various embodiments are described below.

An active electrode assembly 72a of the iontophoresis device 70a employs a porous membrane 78 in place of the buffer material 30 and/or buffer reservoir 32 of the embodiment illustrated in FIG. 1. In particular, the porous membrane 78 is positioned between the inner active electrode ion selective membrane 34 and the outermost ion selective membrane 22. The porous membrane 78 has pores of sufficiently large dimensions as to not be size or molecular weight selective with respect to the particular molecules or compounds contained or produced in or at the active electrode assembly 72a. The porous membrane 78 also is not selective by charge or polarity. Hence, the porous membrane 78 is non-ion selective with respect to the particular application or use.

The porous membrane 78 advantageously spaces the inner active electrode ion selective membrane 34 from the outermost active electrode ion selective membrane 22. By providing one or more spaces between the inner active electrode ion selective membrane 34 and the outermost active electrode ion selective membrane 22, the porous membrane 78 may effectively eliminate or reduce the electrolysis of water which may occur at an interface between the two active electrode ion selective membranes 34, 22. This elimination or reduction in electrolysis may in turn inhibit or reduce the formation of acids and/or bases (e.g., H⁺ ions, OH⁻ ions), that would otherwise present possible disadvantages such as reduced efficiency, reduced transfer rate, and/or possible irritation of the biological interface 18. While illustrated as being non-ion selective, in some embodiments the porous membrane 78 can be replaced with an ion selective membrane.

FIG. 2B shows an iontophoresis device 70b according to another illustrated embodiment, similar to that of FIG. 2A but employing three ion exchange membranes 22a, 22b, 22c to form the outermost active electrode ion selective membrane 22 of the active electrode assembly 72b. As discussed above, the use of multiple ion exchange membranes 22a, 22b, 22c advantageously allows more active agent 24 to be loaded into the active electrode assembly 72b.

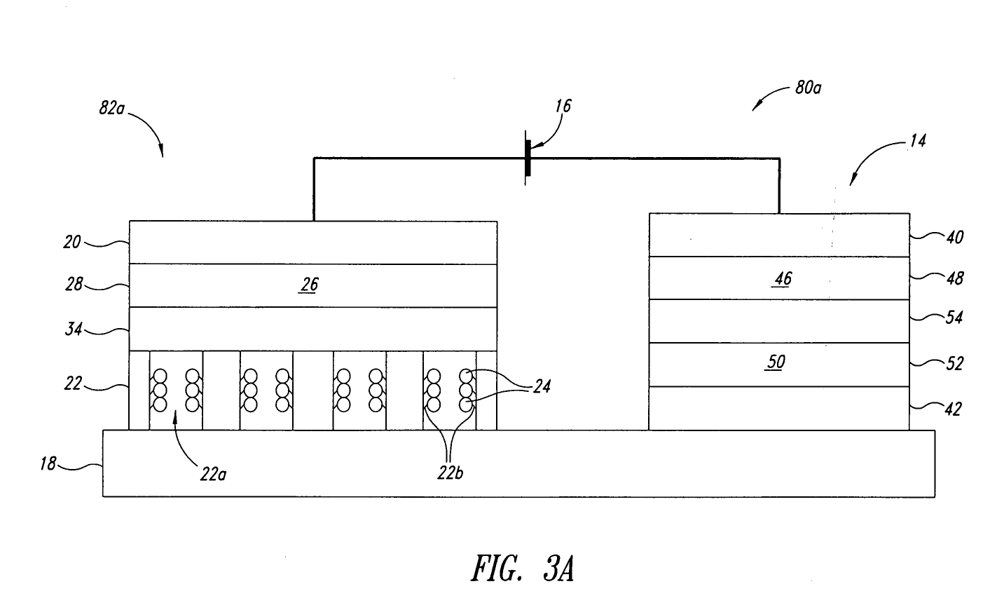

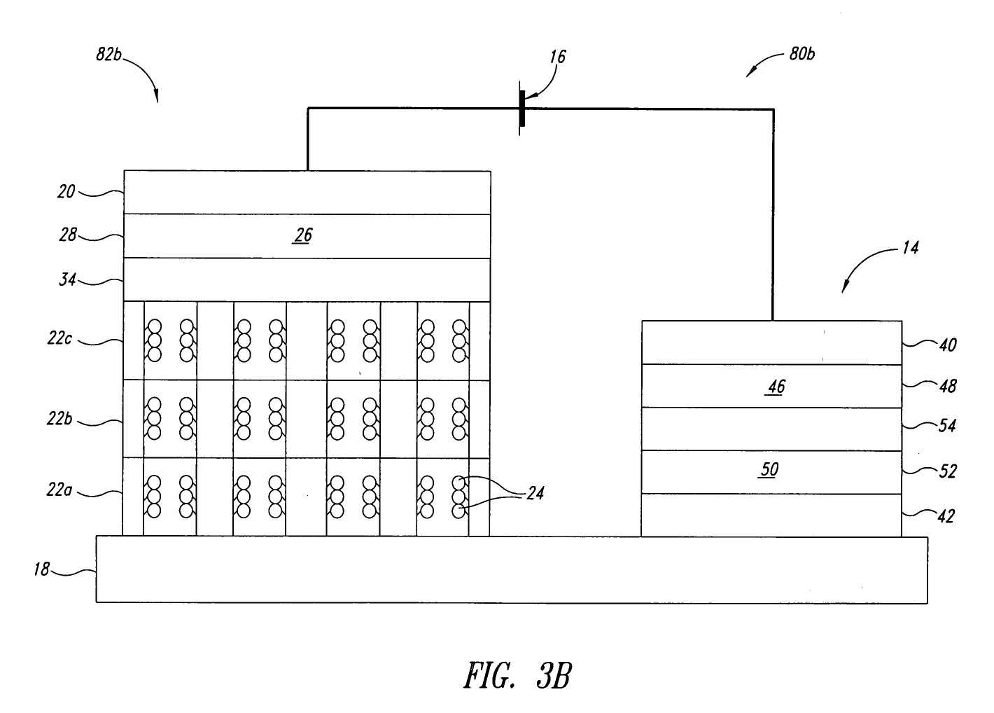

FIG. 3A shows an iontophoresis device 80a according to yet another illustrated embodiment.

The active electrode assembly 82a of the iontophoresis device 80a omits the buffer material 30 and/or buffer reservoir 32 shown in the embodiment illustrated in FIG. 1. Omission of the buffer material 30 and/or buffer reservoir 32 simplifies the iontophoresis device 80a, reducing manufacturing costs and reducing the thickness of the iontophoresis device 80a.

FIG. 3B shows an iontophoresis device 80b according to another illustrated embodiment, similar to that of FIG. 3A but employing three ion exchange membranes 22a, 22b, 22c to form the outermost active electrode ion selective membrane 22 of the active electrode assembly 82b. As noted above, such may permit the loading of a larger dose or quantity of active agent 24 in the active electrode assembly 82b than would otherwise be possible.

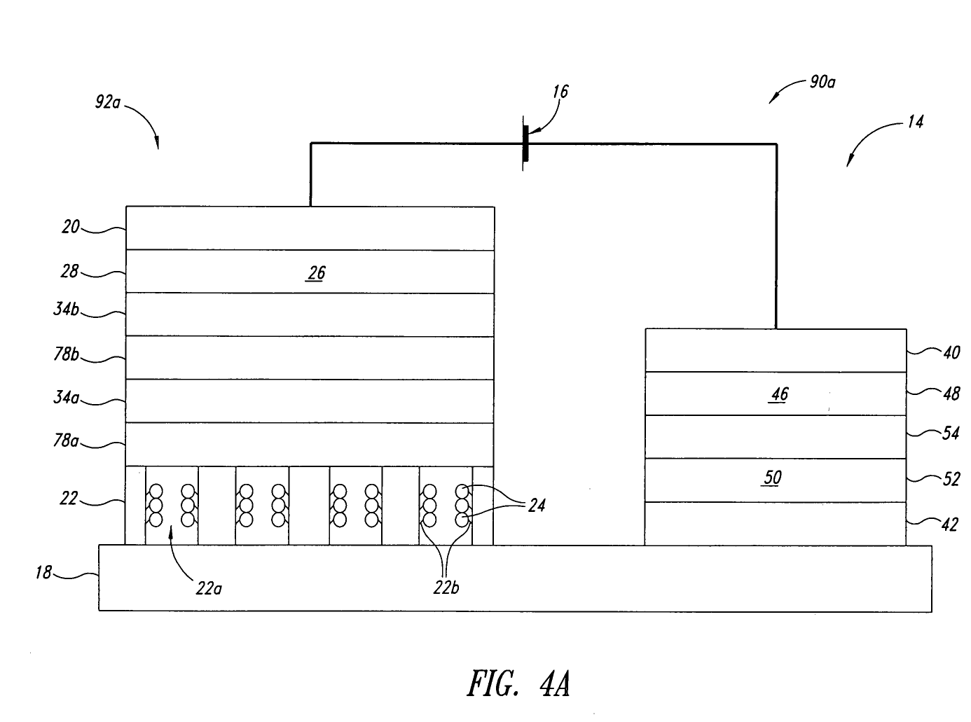

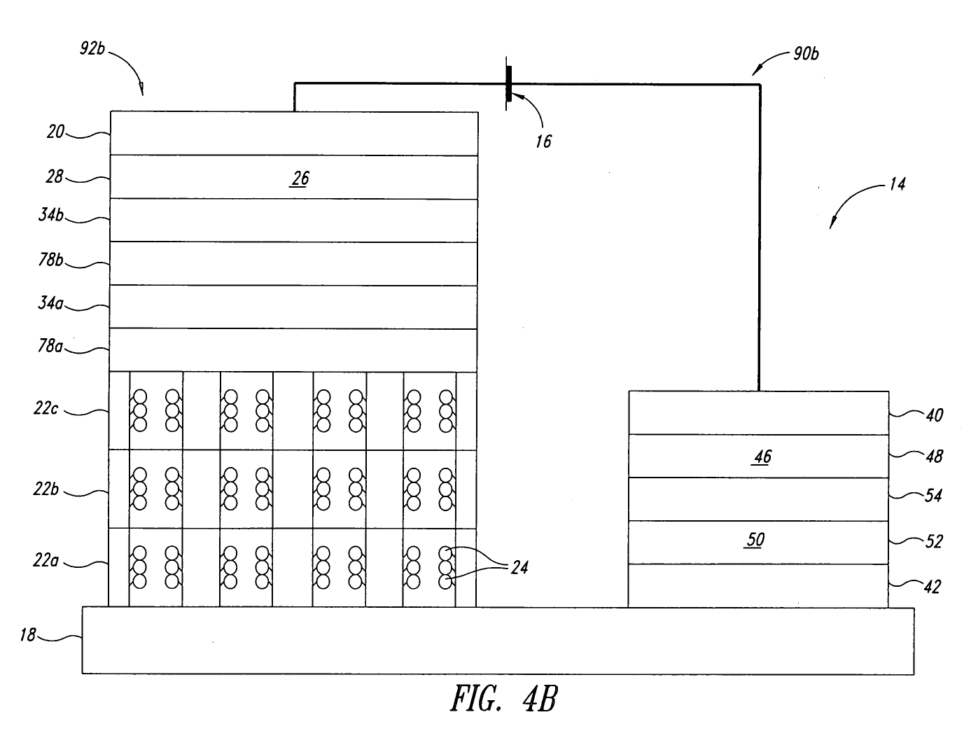

FIG. 4A shows an iontophoresis device 90a according to yet another illustrated embodiment.

The active electrode assembly 92a of the iontophoresis device 90a is similar to that of the embodiment illustrated in FIG. 2, however, the embodiment of FIG. 4 includes first and second inner active electrode ion selective-membranes 34a, 34b. The first inner active electrode ion selective membrane 34a is spaced from the outermost active electrode ion selective membrane 22, for example by a first non-ion selective porous membrane 78a. The second inner active electrode ion selective membrane 34b is spaced from the first inner active electrode ion selective membrane 34a, for example by a second non-ion selective porous membrane 78b. While illustrated as non-ion selective, in some embodiments the first and second non-ion selective porous membranes 78a, 78b may be replaced by ion selective membranes.

FIG. 4B shows an iontophoresis device 90b according to another illustrated embodiment, similar to that of FIG. 4A but employing three ion exchange membranes 22a, 22b, 22c to form the outermost active electrode ion selective membrane 22 of the active electrode assembly 92b. As noted above, such may permit the loading of a larger dose or quantity of active agent 24 in the active electrode assembly 92b than would otherwise be possible.

The above description of illustrated embodiments, including what is described in the Abstract, is not intended to be exhaustive or to limit the claims to the precise forms disclosed. Although specific embodiments of and examples are described herein for illustrative purposes, various equivalent modifications can be made without departing from the spirit and scope of the invention, as will be recognized by those skilled in the relevant art. The teachings provided herein of the invention can be applied to other agent delivery systems and devices, not necessarily the exemplary iontophoresis active agent system and devices generally described above. For instance, some embodiments may include additional structure. For example, some embodiment may include a control circuit or subsystem to control a voltage, current, or power applied to the active and counter electrode elements 20, 40. Also for example, some embodiments may include an interface layer interposed between the outermost active electrode ion selective membrane 22 and the biological interface 18. Some embodiments may comprise additional ion selective membranes, ion exchange membranes, semi-permeable membranes and/or porous membranes, as well as additional reservoirs for electrolytes and/or buffers.

Various electrically conductive hydrogels have been known and used in the medical field to provide an electrical interface to the skin of a subject or within a device to couple electrical stimulus into the subject. Hydrogels hydrate the skin, thus protecting against burning due to electrical stimulation through the hydrogel, while swelling the skin and allowing more efficient transfer of an active component. Examples of such hydrogels are disclosed in U.S. Pat. Nos. 6,803,420; 6,576,712; 6,908,681; 6,596,401; 6,329,488; 6,197,324; 5,290,585; 6,797,276; 5,800,685; 5,660,178; 5,573,668; 5,536,768; 5,489,624; 5,362,420; 5,338,490; and 5,240995, herein incorporated in their entirety by reference. Further examples of such hydrogels are disclosed in U.S. Patent applications 2004/166147; 2004/105834; and 2004/247655, herein incorporated in their entirety by reference. Product brand names of various hydrogels and hydrogel sheets include Corplex™ by Corium, Tegagel™ by 3M, PuraMatrix™ by BD; Vigilon™ by Bard; ClearSite™ by Conmed Corporation; FlexiGel™ by Smith & Nephew; Derma-Gel™ by Medline; Nu-Gel™ by Johnson & Johnson; and Curagel™ by Kendall, or acrylhydrogel films available from Sun Contact Lens Co., Ltd.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety, including but not limited to: Japanese patent application Serial No. H03-86002, filed Mar. 27, 1991, having Japanese Publication No. H04-297277, issued on Mar. 3, 2000 as Japanese Patent No. 3040517; Japanese patent application Serial No. 11-033076, filed Feb. 10, 1999, having Japanese Publication No. 2000-229128; Japanese patent application Serial No. 11-033765, filed Feb. 12, 1999, having Japanese Publication No. 2000-229129, Japanese patent application Serial No. 11-041415, filed Feb. 19, 1999, having Japanese Publication No. 2000-237326; Japanese patent application Serial No. 11-041416, filed Feb. 19, 1999, having Japanese Publication No. 2000-237327; Japanese patent application Serial No. 11-042752, filed Feb. 22, 1999, having Japanese Publication No. 2000-237328; Japanese patent application Serial No. 11-042753, filed Feb. 22, 1999, having Japanese Publication No. 2000-237329; Japanese patent application Serial No. 11-099008, filed Apr. 6, 1999, having Japanese Publication No. 2000-288098; Japanese patent application Serial No. 11-099009, filed Apr. 6, 1999, having Japanese Publication No. 2000-288097; PCT patent application WO 2002JP4696, filed May 15, 2002, having PCT Publication No. W003037425; U.S. patent application Ser. No. 10/488970, filed Mar. 9, 2004; Japanese patent application 2004/317317, filed Oct. 29, 2004; U.S. provisional patent application Serial No. 60/627,952, filed Nov. 16, 2004; Japanese patent application Serial No. 2004-347814, filed Nov. 30, 2004; Japanese patent application Serial No. 2004-357313, filed Dec. 9, 2004; Japanese patent application Serial No. 2005-027748, filed Feb. 3, 2005; and Japanese patent application Serial No. 2005-081220, filed Mar. 22, 2005.

Aspects of the various embodiments can be modified, if necessary, to employ systems, circuits, and concepts of the various patents, applications, and publications to provide yet further embodiments.

These and other changes can be made in light of the above-detailed description. In general, in the following claims, the terms used should not be construed to be limiting to the specific embodiments disclosed in the specification and the claims, but should be construed to include all systems, devices and/or methods that operate in accordance with the claims. Accordingly, the invention is not limited by the disclosure, but instead its scope is to be determined entirely by the following claims.

We/I claim:

1. An iontophoresis device for delivering one or more active agents to a biological interface, comprising:
an-active-electrode element;
an outermost active electrode ion selective membrane spaced from the active electrode element, the outermost active electrode ion selective membrane comprising a plurality of interstices;
one or more active agents of a first polarity loaded in the interstices of the outermost active ionic selective membrane and substantially retained therein in the absence of an electromotive force and transferred outwardly from the outermost active electrode ion selective membrane in the presence of an electromotive force;
an electrolyte positioned between the active electrode element and the outermost active electrode ion selective membrane; and
a first inner active electrode ion selective membrane characterized in that in the presence of an electromotive force of the first polarity, the first inner active electrode ion selective membrane passes ions of a polarity that is opposite to the first polarity and that have a size approximately less than a threshold size, and in that in the presence of the electromotive force of the first polarity the first inner active electrode ion selective membrane limits passage of ions of a polarity that is the same as the first polarity such that a fraction of a total charge flux across the first inner active electrode ion selective membrane that is attributable to the ions of the first polarity is in the range from about 0.05 to about 0.5 of the total charge flux across the first inner active electrode ion selective membrane.

2. The iontophoresis device of claim 1 wherein the first inner active electrode ion selective membrane is further characterized in that in the absence of the electromotive force of the first polarity the fraction of total charge flux across the first inner active electrode ion selective membrane attributable to ions of the first polarity is essentially zero.

3. The iontophoresis device of claim 1 wherein the outermost active electrode ion selective membrane comprises an ion exchange membrane substantially passable by ions of one polarity and-substantially impassable by ions of an opposite polarity.

4. The iontophoresis device of claim 3 wherein the outermost active electrode ion selective membrane comprises a number of ion exchange groups and the active agent is bonded to at least some of the ion exchange groups of the outermost active electrode ion selective membrane in the absence of the electromotive force.

5. The iontophoresis device of claim 1 wherein the active agent is preloaded in the interstices of the outermost active ionic selective membrane before any electromotive force is applied by the active electrode element.

6. The iontophoresis device of claim 1 wherein the outermost active electrode ion selective membrane comprises at least two distinct ion exchange membrane substrates positioned to substantially overlie one another.

7. The iontophoresis device of claim 1 wherein the active agent is a cationic drug and the outermost active electrode ion selective membrane is a cation exchange membrane.

8. The iontophoresis device of claim 1 wherein the active agent is an anionic drug and the outermost active electrode ion selective membrane is an anion exchange membrane.

9. The iontophoresis device of claim 1 wherein the outermost active electrode ion selective membrane is positioned proximate an exterior of the iontophoresis device to make direct contact with the biological interface when in use.

10. The iontophoresis device of claim 1 wherein the first inner active electrode ion selective membrane comprises a cation exchange membrane (CEM) having a pore size sufficiently large as to allow a transfer of anions with a molecular size smaller than the threshold size.

11. The iontophoresis device of claim 1 wherein the first inner active electrode ion selective membrane comprises an anion exchange membrane having a pore size sufficiently large as to allow a transfer of cations with a molecular size smaller than the threshold size.

12. The iontophoresis device of claim 1 wherein the first inner active electrode ion selective membrane is spaced from the outermost active electrode ion selective membrane.

13. The iontophoresis device of claim 1, further comprising:
a porous membrane positioned to space the first inner active electrode ion selective membrane from the outermost active electrode ion selective membrane.

14. The iontophoresis device of claim 1, further comprising:
a second inner ion selective membrane positioned between the first inner ion exchange membrane and the electrolyte;
a first non-ion selective porous membrane spacing the first inner ion selective membrane from the outermost active electrode ion selective membrane; and
a second non-ion selective porous membrane spacing the second inner ion selective membrane from the first inner ion selective membrane.

15. The iontophoresis device of claim 1 wherein the combination of the active electrode element, the electrolyte, the inner active electrode ion selective membrane and the outermost active electrode ion selective membrane retaining the active agent form an active electrode assembly, and further comprising:
a counter electrode assembly; and
a voltage source electrically coupled between the active and the counter electrode assemblies.

16. The iontophoresis device of claim 15 wherein the counter electrode assembly comprises:
a counter electrode element; and
an outermost counter electrode ion selective membrane selectively passing ions of an opposite polarity to that of the outermost active electrode ion selective membrane of the active electrode assembly; and
an electrolyte positioned between the counter electrode element and the outermost counter electrode ion selective membrane.

17. The iontophoresis device of claim 16 wherein the counter electrode assembly further comprises:
an inner counter electrode ion selective membrane positioned between the electrolyte and the outermost counter electrode ion selective membrane; and
a buffer material positioned between the inner counter electrode ion selective membrane and the outermost counter electrode ion selective membrane.

18. The iontophoresis device of claim 1 wherein the electrolyte comprises ions having an identical structure than that of the active agent in the outermost counter electrode ion selective membrane.

19. An iontophoresis device for delivering one or more active agents to a biological interface, comprising:
an active electrode element;
an outermost active electrode ion selective membrane spaced from the active electrode element, the outermost active electrode ion selective membrane comprising a plurality of interstices;
one or more active agents of a first polarity loaded in the interstices of the outermost active ionic selective membrane and substantially retained therein in the absence of an electromotive force and transferred outwardly from the outermost active electrode ion selective membrane in the presence of an electromotive force;
an electrolyte positioned between the active electrode element and the outermost active electrode ion selective membrane; and
a first inner active electrode ion selective membrane characterized in that in the presence of an electromotive force of the first polarity, the first inner active electrode ion selective membrane passes ions of a polarity that is opposite to the first polarity and that have a size approximately less than a threshold size, and in that in the presence of the electromotive force of the first polarity the first inner active electrode ion selective membrane limits passage of ions of a polarity that is the same as the first polarity such that passage of ions of the first polarity from the electrolyte across the first inner active electrode ion selective membrane is approximately equal to or greater than a flux of the one or more active agents of the first polarity across the outermost active electrode ion selective membrane.

20. The iontophoresis device of claim 19 wherein the first inner active electrode ion selective membrane is further characterized in that in the absence of the electromotive force of the first polarity a total flux across the first inner active electrode ion selective membrane attributable to ions of the first polarity is essentially zero.

21. The iontophoresis device of claim 19 wherein the active agent is a cationic drug, the first inner active electrode ion selective membrane is an anion exchange membrane, and the outermost active electrode ion selective membrane is a cation exchange membrane.

22. The iontophoresis device of claim 19 wherein the active agent is an anionic drug, the first inner active electrode ion selective membrane is a cation exchange membrane, and the outermost active electrode ion selective membrane is an anion exchange membrane.

23. The iontophoresis device of claim 19 wherein the electrolyte comprises ions having an identical structure than that of the active agent in the outermost counter electrode ion selective membrane.

24. An iontophoresis device for delivering one or more active agents to a biological interface, comprising:
an active electrode element;
an outermost active electrode ion selective membrane spaced from the active electrode element, the outermost active electrode ion selective membrane comprising a plurality of interstices;
one or more active agents of a first polarity loaded in the interstices of the outermost active ionic selective membrane and substantially retained therein in the absence of an electromotive force and transferred outwardly from the outermost active electrode ion selective membrane in the presence of an electromotive force;
an electrolyte positioned between the active electrode element and the outermost active electrode ion selective membrane; and
a first inner active electrode ion selective membrane characterized in that in the presence of an electromotive force of the first polarity, the first inner active electrode ion selective membrane passes ions of a polarity that is opposite to the first polarity and that have a size approximately less than a threshold size, and in that in the presence of the electromotive force of the first polarity the first inner active electrode ion selective membrane limits passage of ions of a polarity that is the same as the first polarity such that passage of ions of the first polarity from the electrolyte across the first inner active electrode ion selective membrane ranges from about 600 nmol·min⁻¹ to about 3000 nmol·min⁻¹ when a flux of the one or more active agents of the first polarity across the outermost active electrode ion selective membrane ranges from about 600 nmol·min⁻¹ to about 3000 nmol·min⁻¹.

25. The iontophoresis device of claim 24 wherein the first inner active electrode ion selective membrane is further characterized in that in the absence of the electromotive force of the first polarity a total flux across the first inner active electrode ion selective membrane attributable to ions of the first polarity is essentially zero.

26. The iontophoresis device of claim 24 wherein the active agent is a cationic drug, the first inner active electrode ion selective membrane is an anion exchange membrane, and the outermost active electrode ion selective membrane is a cation exchange membrane.

27. The iontophoresis device of claim 24 wherein the active agent is a pharmaceutical composition comprising Lidocaine or a pharmaceutical salt thereof.

28. The iontophoresis device of claim 24 wherein the active agent is an anionic drug, the first inner active electrode ion selective membrane is a cation exchange membrane, and the outermost active electrode ion selective membrane is an anion exchange membrane.

29. The iontophoresis device of claim 24 wherein the electrolyte comprises ions having an identical structure than that of the active agent in the outermost counter electrode ion selective membrane.
